(19) United States
(12) Patent Application Publication
(10) Pub. No.: US 2026/0130830 A1
(43) Pub. Date: May 14, 2026

(54) BIOADHESIVE COMPOSITION

(71) Applicants: INSTITUT STRAUMANN AG, Basel (CH); Biomimetic Innovations Limited, Shannon, Co. Clare (IE)

(72) Inventors: Benjamin PIPPENGER, Hegenheim (FR); Benjamin BELLON PECECNIK, Basel (CH); Ole ZOFFMANN ANDERSEN, Fredericia (DK)

(73) Assignees: INSTITUT STRAUMANN AG, Basel (CH); Biomimetic Innovations Limited, Shannon, Co. Clare (IE)

(21) Appl. No.: 19/140,202

(22) PCT Filed: Dec. 20, 2023
(86) PCT No.: PCT/EP2023/087153

§ 371 Date: Jun. 17, 2025

(30) Foreign Application Priority Data
Dec. 21, 2022 (EP) 22215521.0
May 25, 2023 (EP) 23175473.0

(51) Int. Cl. A61K 6/30; A61K 6/60; A61K 6/838; A61K 6/876; A61L 24/00; A61L 27/42

(52) U.S. Cl. CPC A61K 6/30; A61K 6/60; A61K 6/838; A61K 6/876; A61L 24/0063; A61L 24/0068; A61L 27/425; A61L 27/427; A61L 2430/02; A61L 2430/12

(57) ABSTRACT

A two-component composition including a first component A including water or an aqueous solution, and a second component B including a self-setting adhesive powder including at least a multivalent metal salt and phosphoserine, and at least one monocarbonate selected from the group of sodium carbonate, ammonium carbonate and potassium carbonate, wherein the monocarbonate is present in a concentration between 4 and 12% by weight of component B.

The present invention relates to a composition for preparing a bioadhesive composition. A kit and a method for preparing a bioadhesive composition are also provided.

Medical devices such as implants in general and dental implants in particular are widely used nowadays. They have become an appreciated possibility where hard tissue structures need to be fixed or replaced, e.g., in the case of bone fractures or tooth loss. However, the success of such implants strongly depends on adequate support at the implant site. If the bone mass at said site is insufficient or poor in quality, bone repair and/or bone augmentation becomes a necessity. There are different treatments applied to regain sufficient bone mass, including the use of bone graft materials of different origin, shape and size.

While there are ways to systemically treat the mass and/or strength of the bone, e.g., in osteoporosis, it is still difficult to achieve bone formation in a reliable and controllable manner. However, local bone formation would greatly benefit the adequate treatment of incidents where enhancing the bone volume is only required locally, for example, when placing dental implants.

Methods currently used to repair bone defects include graft materials from different sources. The material is either synthetic or of natural origin. One natural graft material which is employed is autogenous bone. In contrast to bone or bone like material from natural sources (human, animals, plants, algae etc.), autogenous bone material does not trigger a strong immune response and is thus not rejected by the host. However, autogenous bone material requires a second surgery for harvesting the bone increasing the risk of unwanted infection and/or inflammation at this site and significantly increases treatment costs. Further, the removal of bone material leads, at least temporarily, to a weakened structure at this site and causes a painful healing process.

EP2269663A2 discloses a bone graft or biocomposite for treating osseous defects and neogenesis of bone. It is a composite of a biodegradable polymer and granules of beta-tricalciumphosphate, and further comprises an active ingredient embedded in the biodegradable polymer.

WO2010056811 discloses compositions comprising at least tetra calcium phosphate, an effective amount of a compound that is structurally similar to phosphoserine, and can be mixed with an aqueous solution. The compositions provide adhesive and cohesive strength in both wet and dry environments.

WO2016196371 and WO2018060289 disclose compositions having adhesion properties and methods of fixation of an implant to a bone. Said materials have excellent adhesion properties, in particular as soft tissue adhesive, however, they are very dense which has a negative impact on new bone formation.

WO2021092062A1 discloses adhesive compositions including a therapeutic that is released from the composition to treat any number of ailments or conditions or to help accelerate local tissue regeneration or to assist with surgical or therapeutic treatment.

The object of the present invention is to provide a bioadhesive composition that allows a good osseointegration.

The problem is solved by the composition according to claim 1. Further preferred embodiments are subject of dependent claims 2 to 15.

The composition of the present invention relates to a two-component composition comprising a first component A and a second component B. The first component A comprises water or an aqueous solution. The second component B comprises a self-setting adhesive powder and at least one monocarbonate. Said self-setting adhesive powder comprises at least a multivalent metal salt and phosphoserine. The at least one monocarbonate is selected from the group consisting of sodium carbonate, ammonium carbonate and potassium carbonate, and said at least one monocarbonate is present in a concentration between 4 and 12% by weight of component B.

Upon mixing the two components, i.e. component A and component B, a cohesive, viscous paste is formed, which maintains its tacky character until set. At the same time a gas is created which forms a porous scaffold in said paste. This porous paste can be applied for example to stabilize a implant, in particular a dental implant, or to fill a bone defect. One of the primary advantages of the composition according to the present invention is its inherent ability to set and maintain its adhesive character even in aqueous environments, while at the same time resulting in excellent osseointegration due to the porosity created in situ. This allows for example to adhere a dental implant to bone tissue or soft tissue without having a negative impact on the osseointegration.

It has been found out that the selection of the monocarbonate and the amount of the monocarbonate present in the composition according to the present invention is of crucial importance to obtain sufficient porosity in the cured material while at the same time maintaining sufficient stability. For example, a monocarbonate concentration of less than 4% by weight of component B results in the cured material which is too dense to guarantee a good osseointegration and a monocarbonate concentration of for example 16% by weight of component B strongly impacts the mechanical stability of the cured material. In addition, it was surprisingly found that this effect can only be achieved with monocarbonates selected from the group consisting of sodium carbonate, ammonium carbonate and potassium carbonate. In particular, it is worth mentioning that no pores could be obtained with calcium carbonate which is widely used in biological applications. The composition according to the present invention also serves as a mechanically stable scaffold for bone regrowth as well as implant stabilization.

The composition according to the present invention does not only result in an optimal osseointegration by facilitating the ingrowth of vessels and osseous tissue but also serves as a mechanically stable scaffold for these processes.

The composition according to the present invention is particularly useful as a bone restorative composition. By a bone restorative composition is meant a composition that is useful to restore and/or repair bone, such as bone adhesives, bone cements, bone glues, bone putties, bone void fillers, bone replacement compositions, cements and/or adhesives to fix implants (i.e. to stabilize), such as dental implants, to bone tissue or soft tissue. The composition according to the present invention is particularly useful in the treatment of bone defects, since it has both, a porous scaffold and adhesive properties. Furthermore, as the material expands during the hardening process, it is especially beneficial to fill complex bone defects. Directly after mixing the composition flows smoothly and homogeneously and expands in the void and fills the complete volume of the defect. Furthermore, as explained above the material has excellent adhesive properties and therefore, allows a good adhesion to the surrounding bone, soft tissue and/or dental implant materials. Furthermore, it does not only adhere to biological tissues, but also to materials made of metals, ceramic or plastic, and in particular to dental implants made of titanium, a titanium alloy or Y-TZP.

The first component A of the two-component composition comprises water or an aqueous solution. Within the context of the present invention the term aqueous solution means water that additionally comprises an additive such as a salt. Preferably, component A comprises distilled water or an aqueous solution comprising water and saline (0.9% by weight NaCl in water).

The second component B of the two-component composition comprises a self-setting powder. The term “self-setting” refers to the ability of the material to cure and harden as a result of the mixing of the solid and the liquid component. Said self-setting powder comprises at least two different ingredients, that is, a multivalent metal salt and phosphoserine. In addition to the self-setting powder, component B comprises at least one monocarbonate. When in contact with water or the aqueous solution the interaction of said ingredients results in a tacky and adhesive reaction mixture which contains pores.

Thus, the composition according to the present invention is preferably a ready-to-use system. The composition is injectable and can be used in a dual chamber system for simplified and fast filling of bone defects in a minimally invasive manner. With the help of a dual chamber system, this formulation can greatly facilitate clinical applications by reducing surgery time, decreasing the risk of contamination, and ensuring repeatable results.

Preferably, the phosphoserine is present in an amount from 20% to 50% by weight of the self-setting adhesive powder, preferably, 20 to 30% by weight of the self-setting adhesive powder. Such an amount of phosphoserine results in an optimal amount of pores. Furthermore, it was demonstrated that the phosphoserine content allows to tune the porosity and the curing time.

Preferably, the multivalent metal salt is present in an amount from 50 to 90% by weight of the self-setting adhesive powder, preferably, 70 to 80% by weight of the self-setting adhesive powder.

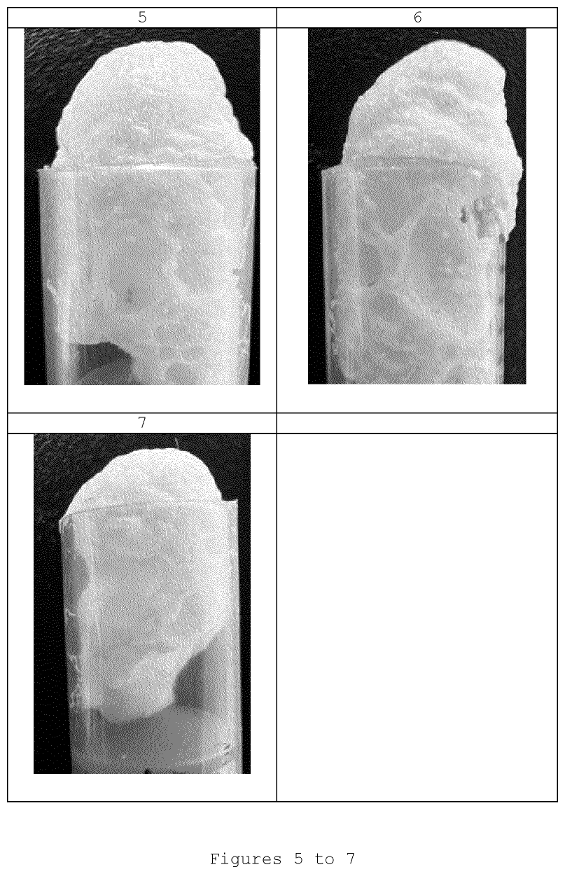

Preferably, the monocarbonate is present in a concentration between 6.5 and 12% by weight of component B. It could be shown that said monocarbonate concentration leads to an optimal result with regard to porosity and mechanical stability (see FIGS. 3 to 5).

Preferably, the water or the aqueous solution of the composition is present in an amount of up to about 35% by weight, preferably up to 25% by weight based on the combined weight of the total composition, i.e. of component A and B together. It was shown that this liquid/solid ratio resulted in an optimal setting time and compressive strength of the adhesive composition.

In one embodiment of the present invention the composition additionally comprises an acidifying agent. In order to control the pore formation, said acidifying agent is preferably part of the first component A, thus, dissolved in water or the aqueous solution. Preferably, said acidifying agent is selected from the group consisting of hydrochloric acid, ascorbic acid, citric acid, malic acid, tartaric acid, maleic acid, succinic acid, fumaric acid, acetic acid, formic acid, propionic acid, their equivalent salts, and in particular trisodium citrate, tripotassium citrate, calcium citrate, magnesium citrate, ammonium citrate and iron citrate, or mixtures thereof, most preferably citric acid, sodium citrate and calcium citrate. The presence of the acidifying agent can be used to adjust the setting time. A higher concentration of the acidifying agent increases the setting time of the material during setting. Preferably, component A comprises 0.1 to 40% by weight of the acidifying agent, most preferably 5% to 25% by weight. Moreover, it has been demonstrated that the acidifying agent, or its equivalent salt, particularly sodium salt, can function as a retarder. Especially good results could be obtained with trisodium citrate.

Preferably, the monocarbonate is selected from the group consisting of sodium carbonate and ammonium carbonate, since they result in an optimal pore formation. Best results could be obtained with sodium carbonate, since compositions comprising sodium carbonate are easier to handle and do not have a tendency to collapse.

In one embodiment of the present invention, the multivalent metal salt contained in the composition comprises tetracalcium phosphate. Tetracalcium phosphate with a comparable particle size distribution results in a slightly faster time and is especially preferred for large bone defects.

In another embodiment of the present invention, the multivalent metal salt contained in the composition comprises tricalcium phosphate, more preferably a-tricalcium phosphate. α-TCP is more soluble in the body's bone material which can increase its absorption rate and shortens the healing process.

Preferably, the phosphoserine in the composition according to the present invention is 1-phosphoserine. L-phosphoserine is a component of many endogenous proteins, in particular osteopontin (bone sialoprotein) and is a normal metabolite found in human biofluids. It has a high affinity for bonding to poorly crystalline apatite, suggesting it plays an important role in mineralization processes.

Preferably, the composition according to the present invention additionally comprises calcium silicate which enhances the setting reaction strength and beneficially influences bone healing and promotes osseointegration.

A further aspect relates to a kit comprising the adhesive composition according to the present invention. Said kit comprises a first compartment with a component A comprising water or an aqueous solution, and a second compartment with component B. Component B comprises a self-setting adhesive powder and at least one monocarbonate selected from the group consisting of sodium carbonate, ammonium carbonate and potassium carbonate, wherein the monocarbonate is present in a concentration between 4 and 12% by weight of component B.

Said self-setting adhesive powder comprises at least a multivalent metal salt and phosphoserine. Said first compartment and said second compartment are physically separated from each other. The separation of the two compartments allows to provide a ready-to-use system having a long shelf life.

The kit comprises preferably 10 to 35% of component A and 65 to 90% of component B, preferably 15 to 20% by weight of component A and 80 to 85% by weight of component B, whereby the total of component A and component B is 100%. It was shown that this liquid/solid ratio resulted in an optimal setting time and compressive strength of the adhesive composition.

The kit comprising the composition according to the present invention can be provided in containers selected from the group consisting of tubes, syringes, bottles, dual barrel syringes, automix systems, titurable capsules, foil packages, and combinations thereof. Such a kit allows an in situ preparation of the material directly before use.

The composition according to the present invention is preferably used in the treatment of bone defects, such as a void, a gap or a crack, so as to fill the bone defect while ensuring mechanical stability during the healing process. Preferably, the bone defect is a large bone defect, since said bone defects are particularly difficult to treat with conventional bone graft materials. Furthermore, the composition also provides a mechanically stable scaffold for bone regrowth as well as implant stabilization. In particular, the composition according to the present invention can be used in oral or dental procedures that requires restoration, e.g., implant placement.

According to a further aspect of the present invention the composition is used in fixing implants, in particular dental implants. Since the composition according to the present invention shows an excellent adhesive affinity for bone, metals and ceramics, the implant is provided with a good primary stability, which allows the implant to heal undisturbed. In addition, the porosity of the cured material results in a good osseointegration. Thus, the composition according to the present invention results in an excellent primary and secondary stability.

A further aspect of the present invention relates to a method for treating bone defects by applying the adhesive composition directly after mixing component A and component B to the site of the bone defect, thereby repairing the bone defect.

According to some embodiments of the invention, the method of repairing a bone defect further includes shaping the composition in the site of the bone defect, which can be done for example with a spatula or with a dedicated delivery system.

According to some embodiments of the invention, the method of repairing a bone defect further includes allowing the composition to set and cure, to thereby form a cured material.

A further aspect of the present invention relates to a method for fixing a dental implant in the site of the missing tooth root by applying the adhesive composition directly after mixing component A and component B to the site of the missing tooth or on the surface of the dental implant and placing the dental implant at the site of the missing tooth.

The cured material is formed by mixing the first component and the second component and allowing the obtained composition sufficient time to set. Initial setting may typically be achieved within 15 seconds and 90 seconds, and final setting may typically be achieved within 1 to 15 minutes. Such short setting times are acceptable since the mixing of the components can be conducted adjacent to or at the site of administration and administration follows immediately after mixing, thereby avoiding delay in surgical procedures owing to long setting times.

In one embodiment of the present invention the first component has a pH of less than 7, preferably less than 4. A pH of less than 4 results in a longer setting time of the composition. In this case, initial setting may be achieved after more than 60 seconds. Such pastes are desirable for larger, load bearing bone defects, where a longer setting time may be required. Said pastes can be manipulated, sculpted and cured in place with immediate high strength capability.

### EXAMPLES

### Material

Self-setting adhesive powder (76% α-TCP (Innotere GmbH), 24% phosphoserine (Merck)

Sodium carbonate (Na₂CO₃) and ammonium carbonate ((NH₄)₂CO₃) Citric acid

### Water (Millipore)

### Experiments

Different ratios of the listed substances were tested to evaluate their impact on the porosity of the cured material. All experiments were performed according to the following protocol:
1) Mix monocarbonate with the self-setting adhesive (the mixing was performed in a plastic vial by shaking thoroughly)
2) Add citric acid solution or pure water
3) Mix for 10 s with a spatula in the plastic vial
4) Fill the mixture in a syringe

In the following table the different ratios can be found. The ratio of the self-setting adhesive powder and the water or the aqueous solution (with or without citric acid) was kept constant.

| | | | | Sodium | |
|---|---|---|---|---|---|
| | Sodium | Self-setting | | monocarbonate in % | Water |
| | monocarbonate | powder | Calculation | of component B | [μl] |
| #1 | 0.01 g | 0.5 g | 0.001 g/0.51 g | 2 | 125 |
| #2 | 0.021 g | 0.5 g | 0.021 g/0.521 g | 4 | 125 |
| #3 | 0.036 g | 0.5 g | 0.036 g/0.536 g | 6.7 | 125 |
| #4 | 0.05 g | 0.5 g | 0.05 g/0.55 g | 9.1 | 125 |
| #5 | 0.065 g | 0.5 g | 0.065 g/0.565 g | 11.5 | 125 |
| #6 | 0.075 g | 0.5 g | 0.075 g/0.575 g | 13 | 125 |
| #7 | 0.085 g | 0.5 g | 0.085 g/0.585 g | 14.5 | 125 |

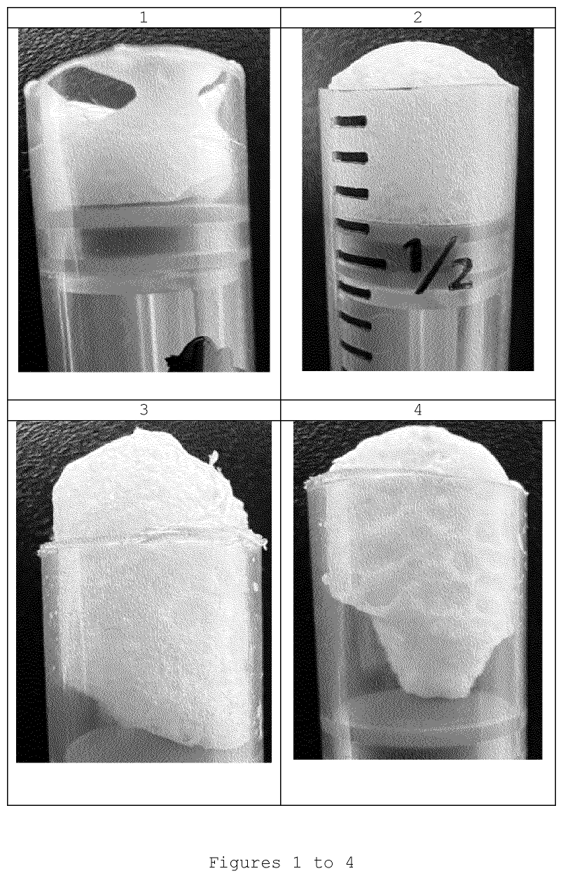

FIGS. 1 to 7 show the impact of the monocarbonate on the porosity of the cured material. In samples 1 and 2 the cured material is too dense, whereas in sample 7 the pores have a negative impact on the mechanical stability. Best results are obtained with samples 3, 4 and 5.

### Example 2: Acidifying Agent

Citric acid solutions of 15 and 30% [w/v] were prepared and the pH of the solutions determined. The pH value of the two solutions is comparable:

$$15 \% [ w / v ] -> pH = 3.3$$

$$30 \% [ w / v ] -> pH = 3.1$$

It was evaluated whether the use of different concentrations of citric acid has an effect on the setting or the bubble formation.

The bubble formation is, compared to experiments with no citric acid, slow and appears to continue for a longer period of time.

It was found that citric acid can influence setting-time and compressive strength of the cured material.

### Example 3: Comparison Sodium Carbonate and Calcium Carbonate

Preparation of self-setting adhesive powder with Na₂CO₃ at 7% [w/w]

Preparation of self-setting adhesive powder with CaCO₃ at 7% [w/w]

Na₂CO₃ results in more (approximately 100% more) volume growth than CaCO₃.

CaCO₃ has no visible pore formation (no gas production)

### Example 4: Impact of Citric Acid on Na₂CO₃ and CaCO₃

Preparation of self-setting adhesive powder with Na₂CO₃ at 7% [w/w] and 15% citric acid.

Preparation of self-setting adhesive powder with CaCO₃ at 7% [w/w] and 15% citric acid.

No bubble formation is observed for the CaCO₃.

The use of citric acid extends the setting time of the material during mixing (compared to when water is used).

For the Na₂CO₃ sample the volume growth appear to be slower, however, the growth continues for a longer period of time.

### Example 5: Carbonates Dissolved in the Liquid Phase

Citric acid was added as powder to the self-setting adhesive powder and the carbonates were dissolved in the water phase prior to mixing the self-setting adhesive powder comprising citric acid.

For Na₂CO₃ the addition of the solution to the self-setting adhesive powder comprising citric acid results in an initial aggressive fizz (within seconds from the solution coming into contact with the powder), after which, there was no more gas development and, hence, no volume growth or pore formation.

For CaCO₃ nothing happened. There was no volume growth or pore formation.

### Example 5: Ammonium Carbonate

Preparation of an equimolar formulation corresponding to the 7% [w/w] Na₂CO₃ formulation

The sample comprising (NH₄)₂CO₃ produced an ammonia smell during the mixing of the components.

(NH₄)₂CO₃ produces even more bubbles than the corresponding Na₂CO₃ sample. The sample comprising (NH₄)₂CO₃ is more difficult to get out of the syringe, as it can collapse when force is applied/plunger is pushed. This finding indicated that the type of carbonate used affects the mechanical properties of the cured material.

### Example 6: Comparison α-TCP vs TTCP

| Formulation | | | Phosphoserine |
|---|---|---|---|
| name | α-TCP | TTCP | ratio [w/w] |
| Alpha24 | 76% | | 24% |
| Tetra24 | | 76% | 24% |
| Alpha36 | 64% | | 36% |
| Tetra36 | | 64% | 36% |

For each experiment, 0.5 g of the self-setting powder and 35 mg sodium carbonate (7% w/w) were mixed with 0.125 ml water.

### Example 6a: Comparison of Alpha24 and Tetra24

35 mg (corresponds to 7% w/w of the powder) of sodium carbonate was added to the powder before mixing for 10 s with water.

The following observations were made:
TTCP sets faster compared to α-TCP (approximately 25%),
overall volume gain/final porosity is higher for α-TCP, presumably due to the slower setting since there is more time for gas expansion/pore formation.

### Example 6b: Influence of Phosphoserine (PS) Content

Two different phosphoserine ratios were prepared for both α-TCP and TTCP (24% and 36%)

7% w/w Sodium carbonate was added to each of the powder formulations before mixing for 10 s with water.

The following observations were made:
a higher phosphoserine content leads to a higher overall volume gain/porosity. This is observed for both TCP's,
the pores seem to be larger for the 36% PS samples compared to 24% PS,
this effect seems to be more pronounced for α-TCP compared to TTCP (presumably due to the slower setting->there is more time for gas expansion/pore formation),
for 36% PS the mixture takes longer to set compared to 24%. Seems like PS itself acts as a retardant. Presumably this is the reason for the higher overall porosity. This effect is more pronounced for α-TCP compared to TTCP.

1. A two-component composition comprising
a first component A comprising water or an aqueous solution, and a second component B comprising
1) a self-setting adhesive powder comprising at least a multivalent metal salt and phosphoserine, and
2) at least one monocarbonate selected from the group consisting of sodium carbonate, ammonium carbonate and potassium carbonate,
wherein the monocarbonate is present in a concentration between 4 and 12% by weight of component B.

2. The composition according to claim 1, wherein the monocarbonate is present in a concentration between 6.5 and 12% by weight of component B.

3. The composition according to claim 1, additionally comprising an acidifying agent selected from the group consisting of hydrochloric acid, ascorbic acid, citric acid, malic acid, tartaric acid, maleic acid, succinic acid, fumaric acid, acetic acid, formic acid, propionic acid, their corresponding salts or mixtures thereof.

4. The composition according to claim 3, wherein the acidifying agent is part of the first component A.

5. The composition according to claim 4, wherein component A comprises 0.1 to 40% by weight of the acidifying agent.

6. The composition according to claim 1, wherein the monocarbonate is selected from the group consisting of sodium carbonate and ammonium carbonate.

7. The composition according to claim 1, wherein the multivalent metal salt comprises tetracalcium phosphate.

8. The composition according to claim 1, wherein the multivalent metal salt comprises tricalcium phosphate.

9. The composition according to claim 1, wherein the self-setting adhesive powder of component B additionally comprises calcium silicate.

10. A kit for preparing a composition according to claim 1, comprising
a first compartment with a component A comprising water or an aqueous solution, and
a second compartment with component B comprising
1) a self-setting adhesive powder comprising at least a multivalent metal salt and phosphoserine,
2) at least one monocarbonate selected from the group consisting of sodium carbonate, ammonium carbonate and potassium carbonate,
wherein the monocarbonate is present in a concentration between 4 and 12% by weight of component B, and wherein the first compartment and the second compartment are physically separated from each other.

11. The kit according to claim 10, comprising 10 to 35% of component A and 65 to 90% of component B, whereby the total of component A and component B is 100%.

12. The composition according to claim 1 for use to restore or repair bone.

13. The composition according to claim 12 for use in the treatment of bone defects.

14. The composition according to claim 1 for use to stabilize implants.

15. The composition according to claim 14 for use to stabilize a dental implant to bone tissue or soft tissue.
